(19) United States
(12) Patent Application Publication
(10) Pub. No.: US 2022/0024904 A1
(43) Pub. Date: Jan. 27, 2022

(54) REAGENT FOR BIOCONJUGATION VIA IRREVERSIBLE REBRIDGING OF DISULFIDE LINKAGES

(71) Applicants: Research Foundation of the City University of New York, New York, NY (US); University of Saskatchewan, Saskatoon (CA)

(72) Inventors: Brian Zeglis, New York, NY (US); Guillaume Dewaele-Le Roi, New York, NY (US); Eric W. Price, Saskatoon (CA); Elaheh Khozeimeh Sarbisheh, Saskatoon (CA)

(21) Appl. No.: 17/368,272

(22) Filed: Jul. 6, 2021

Related U.S. Application Data
(60) Provisional application No. 63/048,353, filed on Jul. 6, 2020.
(60) Provisional application No. 63/216,672, filed on Jun. 30, 2021.

(51) Int. Cl. C07D 413/10; A61K 49/00; A61K 51/10; A61K 51/04; A61K 47/68; A61K 47/54

(52) U.S. Cl. CPC C07D 413/10; A61K 49/0058; A61K 49/0043; A61K 51/1045; A61K 47/547; A61K 51/1093; A61K 47/6889; A61K 47/6851; A61K 51/0482

(57) ABSTRACT

A label that permits rebridging of disulfide linkages in antibodies or proteins. The label has a general formula given by

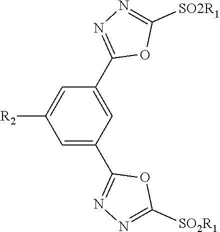

wherein R₁ is methyl, ethyl or propyl and R₂ is a metal chelator, a fluorophore or a click-chemistry synthon.

### CROSS-REFERENCE TO RELATED APPLICATIONS

This application claims priority to, and is a non-provisional of, U.S. provisional patent applications 63/048,353 (filed Jul. 6, 2020) and 63/216,672 (filed Jun. 30, 2021) the entirety of which are incorporated herein by reference.

### STATEMENT OF FEDERALLY SPONSORED RESEARCH OR DEVELOPMENT

This invention was made with government support under grant numbers R01CA240963; R01CA204167 and U01CA221046 awarded by the National Institute of Health. The government has certain rights in the invention.

### BACKGROUND OF THE INVENTION

Over the last two decades, immunoconjugates have emerged as vitally important therapeutic and diagnostic tools. However, the imprecise synthetic methods used to create many antibody-drug conjugates (ADCs) and radioimmunoconjugates remains an impediment to their widespread success. Traditional approaches to bioconjugation are predicated on the indiscriminate attachment of payloads—e.g., chelators, fluorophores, or toxins—to lysine residues within antibodies. Yet these non-site-specific synthetic strategies inevitably lead to heterogeneous product mixtures and can produce constructs with suboptimal immunoreactivity and in vivo performance.

In light of these issues, the development of “site-specific” bioconjugation methods designed to append cargoes only at well-defined sites within an antibody's macromolecular structure has become an area of intensive research. A wide variety of these approaches have been devised, including variants based on the manipulation of the heavy chain glycans, the use of peptide tags, and the genetic incorporation of unnatural amino acids. Far and away the most popular methods, however, rely upon the reaction between maleimide-based bifunctional probes and cysteine residues within the biomolecule (FIG. 1A). While maleimide-based bioconjugation strategies are undeniably facile, rapid, and modular, they nonetheless suffer from a critical flaw: the inherent instability of the thioether bond between the maleimide and the cysteine. The Michael addition reaction that forms this linkage is reversible in vivo both spontaneously (retro-Michael) and in the presence of competing thiols. This, of course, can be a significant problem. In the context of radioimmunoconjugates, for example, this process can result in the in vivo release of radionuclides, reducing target-to-background activity concentration ratios and increasing radiation doses to healthy tissues.

In an effort to circumvent the inherent limitations of maleimides, the synthesis, characterization, and in vivo validation of an alternative, phenyloxadiazolyl methylsulfone or “PODS”, was developed. PODS is an easily synthesized reagent capable of rapidly and irreversibly forming covalent linkages with thiols (FIG. 1B). This work clearly illustrated that a ⁸⁹Zr-DFO-labeled variant of the huA33 antibody synthesized using a PODS-based bifunctional chelator exhibited superior in vitro stability and, even more importantly, in vivo performance compared to an analogous radioimmunoconjugate synthesized using a traditional, maleimide-based probe. Furthermore, the innate modularity of PODS enabled the creation of PODS-CHX-A″-DTPA and PODS-DOTA bifunctional chelators for the synthesis of radioimmunoconjugates labeled with lutetium-177 and actinium-225.

While PODS-based reagents represent a distinct improvement compared to their maleimide-based forerunners, neither tool can avoid an intrinsic problem common to the overwhelming majority of thiol-targeted bioconjugations. In the absence of free cysteine residues incorporated via genetic engineering, all of the cysteines within an antibody are paired to form 8 intrachain and 8 interchain disulfide bridges. As a result, thiol-based bioconjugation strategies require the reduction of these disulfide bridges to generate free thiols, with the slightly easier-to-reduce interchain linkages often the target of selective scission. While the subsequent reaction of these free cysteines with thiol-selective probes enables the site-specific attachment of cargoes to the immunoglobulin, it simultaneously seals the fate of the broken disulfide bridges, potentially reducing the stability of the macromolecule and attenuating effector functions. A handful of reagents capable of reacting with two thiols and thus reforming the covalent bridge between the reduced cysteine residues have been developed. However, immunoconjugates synthesized using the most widely studied of these tools—dibromo- and dithiophenolmaleimides—are still prone to instability in vivo. While the developers of this “next generation maleimide” technology tout this reversibility as an advantage in the context of ADCs, it nonetheless remains an obstacle for radio-immunoconjugates.

The discussion above is merely provided for general background information and is not intended to be used as an aid in determining the scope of the claimed subject matter.

### SUMMARY

This disclosure provides a label that permits rebridging of disulfide linkages in antibodies or proteins. The label has a general formula given by

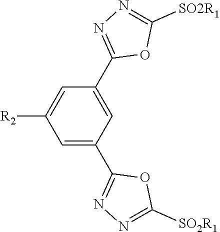

wherein R1 is methyl, ethyl or propyl and R₂ is a metal chelator, a fluorophore or a click-chemistry synthon.

In a first embodiment, a composition of matter is provided. The composition consisting of:

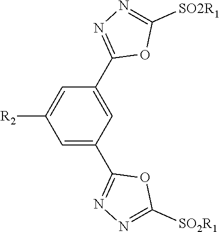

wherein R₁ is methyl, ethyl or propyl and R₂ is a fluorescent label, a metal chelator or a click-chemistry synthon.

In a second embodiment, a method for labeling a substrate is provided. The method comprises steps of: exposing a label to a substrate that comprises two cysteine residues, wherein the label comprises:

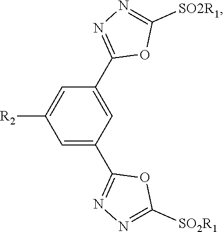

wherein R₁ is methyl, ethyl or propyl and R₂ is a fluorescent label, a metal chelator or a click-chemistry synthon; permitting the label to covalently bind to the two cysteine residues of the substrate, thereby labeling the substrate.

In a third embodiment, a composition of matter is provided. The composition consisting of:

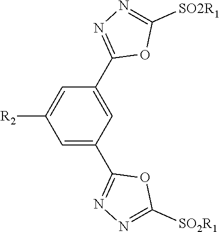

wherein R₁ is methyl and R₂ is a fluorescent label, a metal chelator or a click-chemistry synthon.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A depicts a reaction between maleimide-based bifunctional probes and cysteine residues within a biomolecule;

FIG. 1B depicts PODS, a reagent capable of rapidly and irreversibly forming covalent linkages with thiols;

FIG. 1C depicts DiPODS, a reagent capable of rebridging disulfide linkages;

FIG. 2A is a schematic depiction of the reduction of Fab_{HER2} followed by treatment with a DiPODS variant;

FIG. 2B and FIG. 2C are images of a gel electrophoresis demonstrating the stability of the rebriding;

FIG. 2D and FIG. 2D and FIG. 2E show the results of flow cytometry analysis of HER2-positive BT474 human breast cancer cells stained with Fab_{HER2}-DiPODS-FITC (FIG. 2D) and Fab_{HER2}-Lys-FITC (FIG. 2E);

FIG. 3A shows the treatment of a reduced Fab_{HER2} with DiPODS-NOTA;

FIG. 3B is an image of a gel electrophoresis showing DiPODS-NOTA successfully binds with the reduced Fab_{HER2};

FIG. 4 is a scheme showing the DiPODS-NOTA variant binds to various radiolabels;

FIG. 5 is a graph depicting the binding fraction of several DiPODS-NOTA variant compared to conventional lysine binding methods;

FIG. 6A shows iTLC radioanalysis of [68Ga]-DiPODS-NOTA-Fab_{HER2} while FIG. 6B shows a corresponding analysis of [64Cu]-DiPODS-NOTA-Fab_{HER2};

FIG. 7A shows a SEC-HPLC radioanalysis of [68Ga]-DiPODS-NOTA-Fab_{HER2} by UV-detection while FIG. 7B shows a corresponding analysis of the same compounds by radio-detection;

FIG. 8A shows a SEC-HPLC radioanalysis of [64Cu]-DiPODS-NOTA-Fab_{HER2} by UV-detection while FIG. 8B shows a corresponding analysis of the same compounds by radio-detection;

FIG. 9A is a graph showing human serum stability of [64Cu]-DiPODS-NOTA-Fab_{HER2} over a four hour time frame;

FIG. 9B is a graph depicting the stability of 64[Cu]-DiPODS-NOTA-Fab_{HER2} in human serum over four hours;

FIG. 9B and FIG. 9C are radiochemical purity (%) assays of the same compound monitored by radio-iTLC at t=0 min (FIG. 9B) and at t=4 h (FIG. 9C) directly after incubation in human serum. The consistent results over the observed time period demonstrates the stability of the bioconjugate;

FIG. 10A is a graph depicting the stability of 68[Ga]-DiPODS-NOTA-Fab_{HER2} in human serum while FIG. 10B and FIG. 10C are radiochemical purity (%) assays of the same compound monitored by radio-iTLC at t=0 min (FIG. 10B) and at t=2 h (FIG. 10C) directly after incubation in human serum;

FIG. 11A and FIG. 11B depict one synthetic scheme for the production of DiPODS;

FIG. 12A is an alternative synthesis of compound 6;

FIG. 12B depicts modifying the amine nucleophilic group to a carboxylic acid group;

FIG. 12C shows modification of peptide coupling reagent to use EEDQ;

FIGS. 13A to 13E shows ¹H-NMR spectra of various conformers of a DiPOD;

FIG. 14 depicts spatial interactions in various the conformers;

FIG. 15 is a graphic showing relative energy of different conformers;

FIG. 16 is a graphic showing the energy of the transition state between two conformers: anti-1b and syn-1b;

FIG. 17A and FIG. 17B show the results of computational methods with regard to a bivalent maleimide (FIG. 17A) in comparison to a DiPODS (FIG. 17B);

FIG. 18 is a synthetic scheme for attaching various tags; and

FIG. 19 is a scheme showing the addition to a fluorophore to the primary amine of a DiPODs.

### DETAILED DESCRIPTION OF THE INVENTION

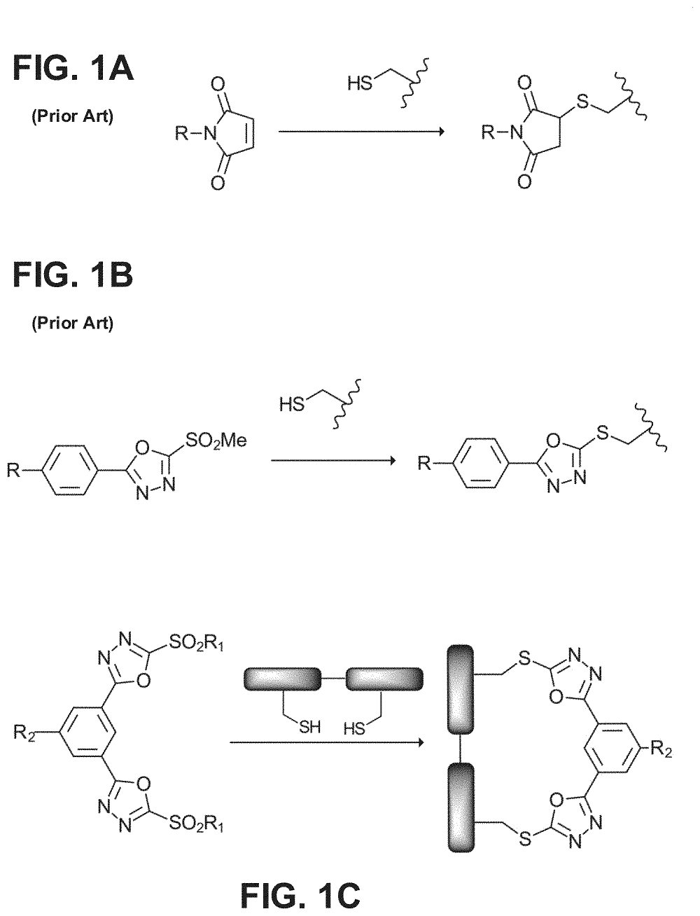

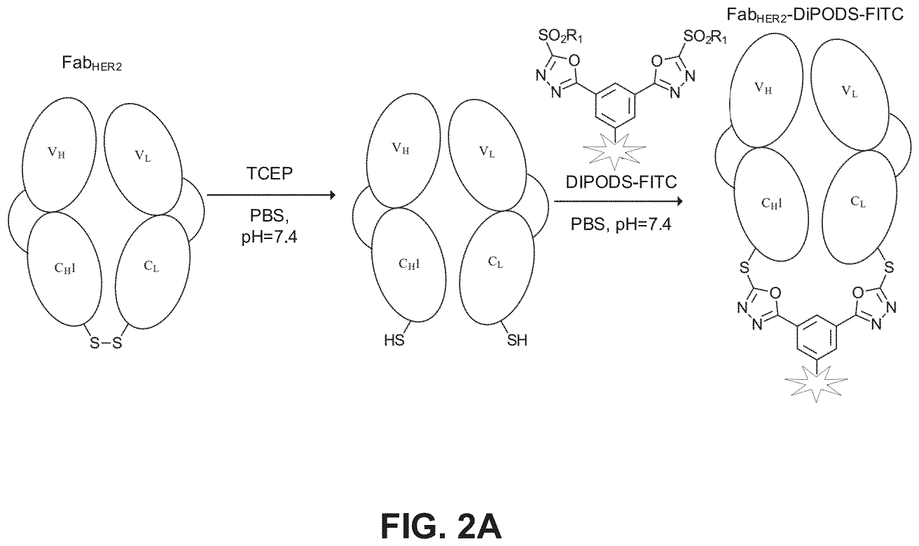

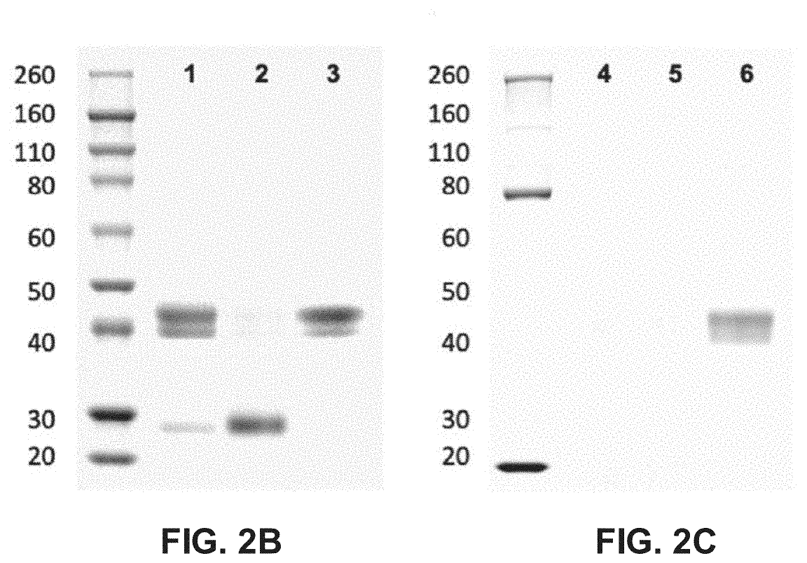

This disclosure provides DiPODS, a novel reagent bearing two oxadiazolyl methyl sulfone moieties designed to provide a modular platform for irreversible bioconjugations while simultaneously rebridging disulfide linkages (FIG. 1C). In FIG. 1C, R₁ is methyl, ethyl or propyl and R2 may be any suitable label such as a fluorescent label, a radiolabel, a click-chemistry synthon and the like.

Examples of suitable fluorescent labels include fluorescein, NHS-Fluorescein, SCN-Fluorescein (FITC), antibody-based fluorescent labels such at the label sold under the brand name ALEXA FLUOR® 350-750, green fluorescent dyes such as the dye sold under the brand names BODIPY® FL, SCN-BODIPY®, Pacific Blue/Green/Orange, Cyanine 5/5.5n, NHS-Rhodamine, Tetramethylrhodamine-isothiocyanate (TRITC), Texas Red, NHS-Coumarin and SCN-Coumarin, NHS-Oregon Green and SCN-Oregon Green. In one embodiment, the fluorescent dye is an amine-reactive dye that contains N-hydroxysuccinimide (NHS) or isothiocyanate (NCS).

Examples of suitable radiolabels include metal bound by chelators such as tetrazine, 1,4,7-triazacyclononane-N,N′,N″-triacetic acid (NOTA), 1,4,7-triazacyclononane,1-glutaric acid-4,7-acetic acid (NODAGA), 1,4,8,11-tetraazacyclotetradecane-N,N′,N″,N′″-tetraacetic acid (TETA), 1,4,7,10-Tetraazacyclododecane-1,4,7,10-tetraacetic acid (DOTA), 1,4,8,11-Tetraazabicyclo[6.6.2]hexadecane-4,11-diacetic acid (CB-TE2A), diethylenetriaminepentaacetic acid (DTPA), hydroxybenzyl ethylenediamine (HBEd), and 1,8-Diamino-3,6,10,13,16,19-hexaazabicyclo[6,6,6]-eicosane (DiamSar).

Examples of suitable click-chemistry synthons include trans-cycloctyl (TCO) derivatives and N3.

In one embodiment, a fluorescein-labeled variant of the reagent (DiPODS-FITC). In an exemplary embodiment, the reaction conditions for DiPODS-FITC were optimized using both isotype-control and HER2-targeting Fab fragments, and the FITC-bearing immunoconjugates were characterized via gel electrophoresis, size exclusion HPLC, and circular dichroism spectroscopy. Finally, the cell binding behavior of the HER2-targeting Fab-DiPODS-FITC was interrogated via flow cytometry and compared to that of an analogous Fab-FITC immunoconjugate created via a traditional, stochastic lysine-based approach to bioconjugation.

This disclosure also provides a radiolabeled variant of the reagent (DiPODS-NOTA). Surprisingly, the radio labeled antibody fragments bind to their targets better than radiolabeled antibody fragments synthesized using traditional methods.

Fluorescein-Labeled Variant

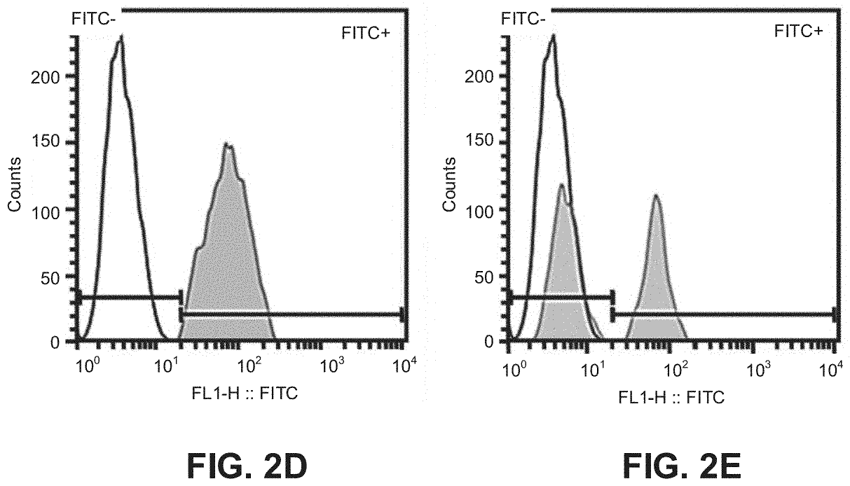

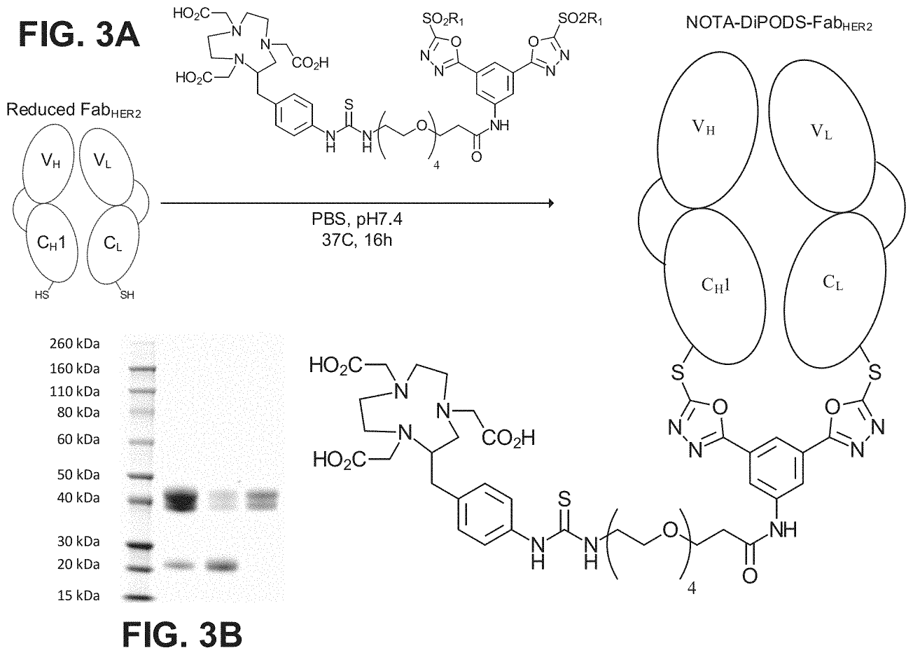

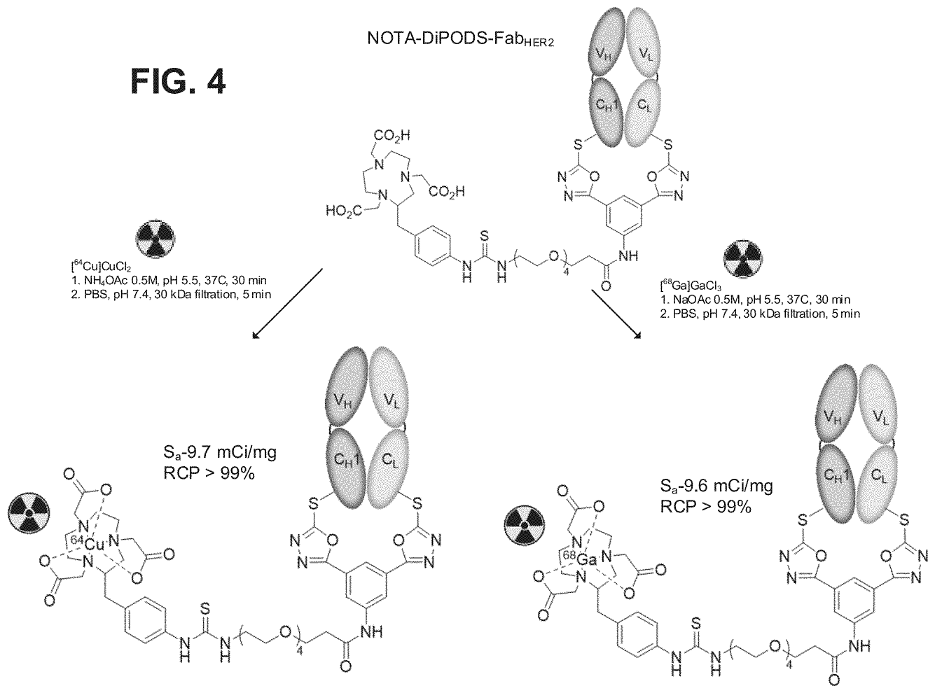

Bioconjugation and Characterization. Fab fragments—rather than full-length IgGs—were selected for proof-of-concept bioconjugation experiments with DiPODS-FITC because of the presence of only a single interchain disulfide linkage (rather than 8) dramatically simplifies the analysis of the products. In practice, two Fabs were employed: a commercially available, nonspecific Fab based on human plasma IgG (Fabₙₛ) and a HER2-targeting Fab created via the enzymatic digestion of trastuzumab (Fab_{HER2}). In each case, the Fab was first treated with TCEP to reduce the interchain disulfide bridge and then incubated with DiPODS-FITC (FIG. 2A). Ultimately, the following optimal reaction conditions were identified: 2 h at 37° C. with 20 equiv of TCEP followed by 16 h with 15 equiv of DiPODS-FITC at the same temperature. Subsequently, UV-vis spectrophotometry was used to measure the degree of labeling (DOL) of each immunoconjugate, revealing that Fabₙₛ-DiPODS-FITC and Fab_{HER2}-DiPODS-FITC were modified with 0.86±0.02 and 0.95±0.01 FITC/Fab, respectively (Table 1). MALDI-TOF mass spectrometry confirmed a degree of labeling of ˜1 for each fluorophore-modified Fab.

TABLE 1
Bioconjugation Results Obtained Using DiPODS-FITC in
Conjunction with Fab_{HER2} and Fabₙₛ

| sample | HS-/Fab ratio | FITC/Fab ratio |
|---|---|---|
| parent Fabₙₛ | undetected | — |
| reduced Fabₙₛ | 2.01 ± 0.12 | — |
| Fabₙₛ-DIPODS-FITC | undetected | 0.86 ± 0.02 |
| parent Fab_{HER2} | undetected | — |
| Reduced Fab_{HER2} | 1.94 ± 0.10 | — |
| Fab_{HER2}-DIPODS-FITC | undetected | 0.95 ± 0.01 |

The stepwise progress of the bioconjugation procedure was monitored using both gel electrophoresis and Ellman's reagent, a chemical tool for the detection of free thiols. In the case of Fab_{HER2}, for example, the former illustrates the decoupling of the intact fragment's VHCH1 and VLCL chains upon reduction with TCEP (FIG. 2B lanes 1 and 2) and the subsequent reunification of the two domains after treatment with DiPODS-FITC (FIG. 2B, lane 3). The analysis of the gel using a fluorescence imager reveals only a single fluorescent band corresponding to an intact, 40-50 kDa Fab and does not show any multimeric cross-bridged species (i.e., Fab-DiPODS-Fab) (FIG. 2C). The use of Ellman's reagent to assess the number of free thiols present at different points of the procedure reinforced the quantitative nature of the approach. The purified Fab_{HER2} starting material contains no detectable free thiols. Reduction with TCEP creates the expected maximum of 1.94 t 0.11 thiols/Fab, a value which went back to effectively zero upon cross-bridging with DiPODS-FITC (Table 1). Importantly, both analytical techniques provided similar results for the bioconjugation of Fabₙₛ.

Next, circular dichroism (CD) spectroscopy was employed to interrogate the structure and melting point of Fab_{HER2}, reduced Fab_{HER2}, and Fab_{HER2}-DiPODS-FITC. Generally speaking, the spectra—which exhibit a positive peak around 205 nm and shallow negative peak around 217 nm—are characteristic of a protein rich in β-sheet content, consistent with the known secondary structure of Fab fragments. The data suggest that the trio of constructs have similar overall structures: the far-UV CD spectra of all three samples have the same shape profile, with only minor differences in ellipticity values which may reflect local conformational adjustments due to the reduction or rebridging of the disulfide bonds. Importantly, the CD data also indicate that the three fragments also share similar thermal stability, as the melting temperatures for Fab_{HER2}, reduced Fab_{HER2}, and Fab_{HER2}-DiPODS-FITC are 65.5, 66.8, and 64.5° C., respectively, when monitored at 205 nm.

Finally, in order to assess the serum stability of Fabₙₛ-DiPODS-FITC and Fab_{HER2}-DiPODS-FITC, the fragments were incubated in 50% human serum albumin (HSA) for 7 days at 37° C. Size exclusion HPLC of each fluorophore-bearing fragment after 7 days yielded a single, unchanged peak). Neither aggregates nor separate V_{H}C_{H1}/V_{L}C_{L} chains nor free fluorophores could be observed, underscoring the stability of the FITC-modified immunoconjugates and the irreversibility of the DiPODS linkage.

In Vitro Evaluation.

With the chemical characterization of Fab_{HER2}-DiPODS-FITC complete, the next step was to ensure that the immunoconjugate retained its ability to bind its molecular target. To this end, flow cytometry experiments were performed using two human breast cancer cell lines: HER2-positive BT474 cells and HER2-negative MDA-MB-235 cells. As a point of comparison, a non-site-specifically modified, HER2-targeting immunoconjugate (Fab_{HER2}-Lys-FITC) was synthesized using a traditional lysine-based approach to bioconjugation and used alongside Fab_{HER2}-DiPODS-FITC in all cell cytometry experiments. The in vitro experiments clearly confirm the specificity of both immunoconjugates, as binding was observed with HER2-positive BT474 cells but not HER2-negative MDA-MB-231 cells. Just as important, however, are the differences between the behavior of the two FITC-modified Fabs and HER2-positive BT474 cells. Under identical conditions—i.e., concentration of cells, concentration of fragments, incubation time—only a single population of fluorophore-positive cells were detected after incubation with Fab_{HER2}-DiPODS-FITC, but both fluorophore-positive and fluorophore-negative cells were observed after incubation with Fab_{HER2}-Lys-FITC (FIG. 2E and FIG. 2E).

These data indicate that the immunoroeactivity of Fab_{HER2}-DiPODS-FITC is higher than that of Fab_{HER2}-Lys-FITC, most likely because the heterogeneous mixture of products that comprises the latter includes immunoconjugates in which fluorophores have been inadvertently appended to the antigen-binding domain of the fragment. These data serve as a reminder that the benefits of site-specific bioconjugation extend beyond simply producing better-defined and more homogeneous immunoconjugates.

These data indicate that the immunoroeactivity of Fab_{HER2}-DiPODS-FITC is higher than that of Fab_{HER2}-Lys-FITC, most likely because the heterogeneous mixture of products that comprises the latter includes immunoconjugates in which fluorophores have been inadvertently appended to the antigen-binding domain of the fragment. These data serve as a reminder that the benefits of site-specific bioconjugation extend beyond simply producing better-defined and more homogeneous immunoconjugates.

Radiolabeled Variant

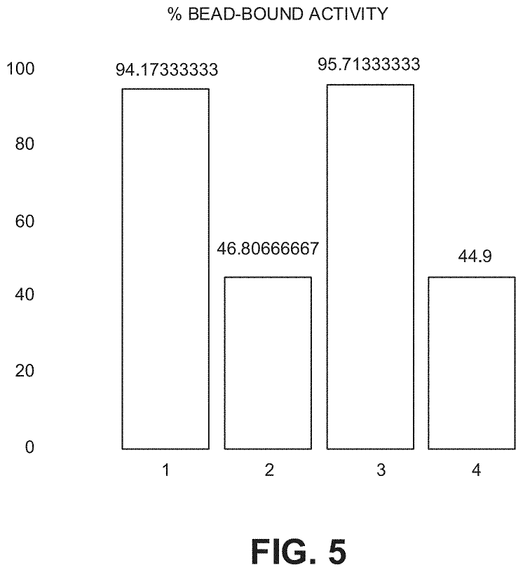

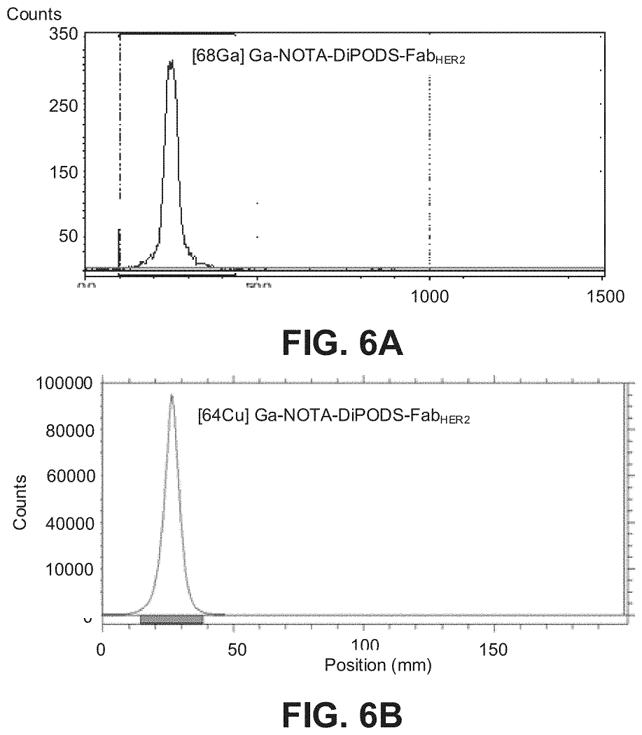

FIG. 3A depicts the use of DiPODS-NOTA to bind to a reduced Fab_{HER2}. FIG. 3B depicts an image from an SDS-PAGE analysis with SIMPLYBLUE™ staining. Lane 1 shows Fab_{HER2} while lane 2 shows reduced Fab_{HER2}. Lane 3 shows the Fab_{HER2}-DiPODS-NOTA complex. Notably, the monomeric forms are absent in lane 3 which evidences the stability of the resulting bioconjugate.

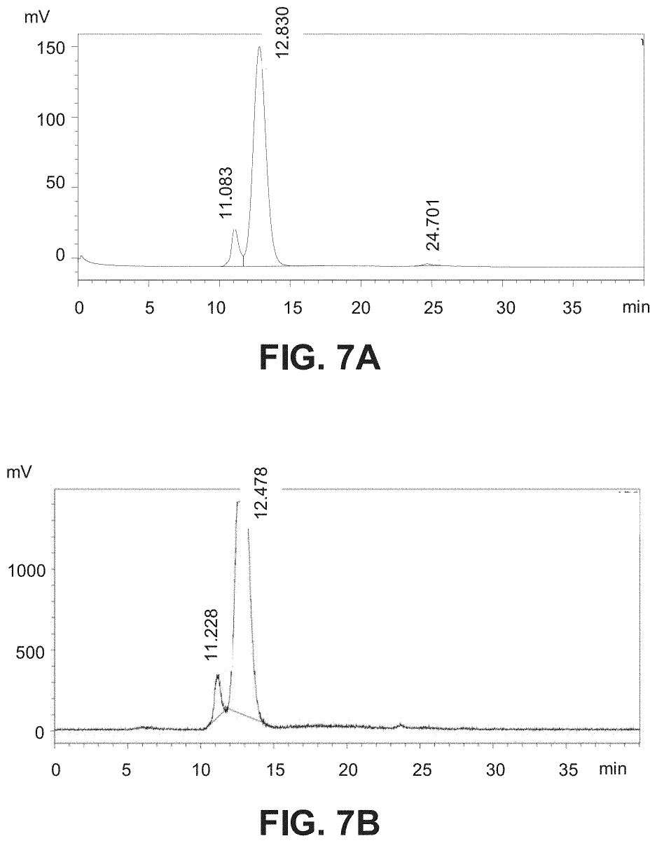

As shown in FIG. 4, this NOTA bioconjugate can be bound to various radiolabels, such as Cu-64 and Ga-68. For example, after binding to the target, the DiPODS-NOTA can be treated with a Cu-64 salt to produce [64Cu]-DiPODS-NOTA. Likewise, after binding to the target, the DiPODS-NOTA can be treated with a Ga-68 salt to produce [68Ga]-DiPODS-NOTA.

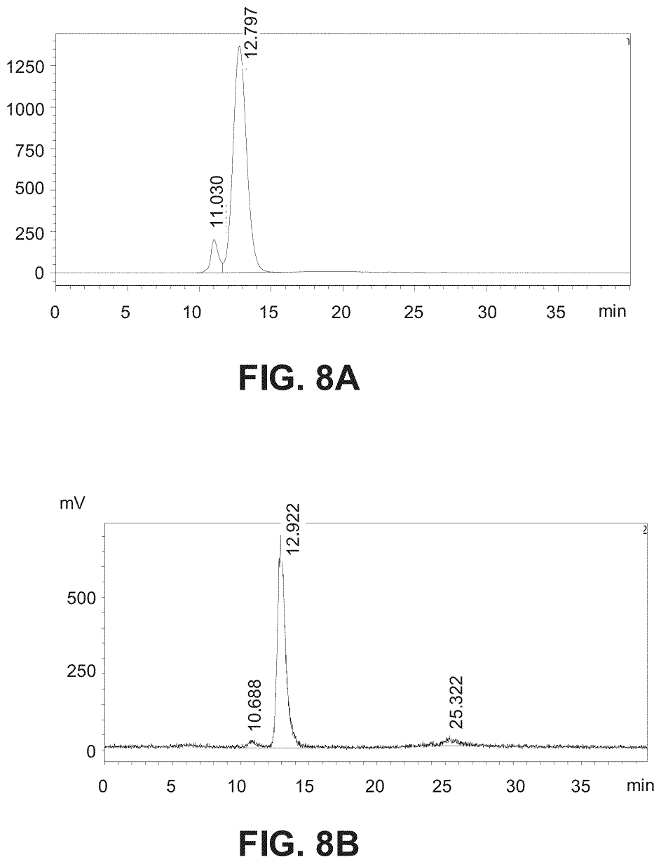

FIG. 5 shows the target-binding fraction of select complexes. Column 1 depicts the target-binding fraction of [64Cu]-DiPODS-NOTA-Fab_{HER2}. Column 2 depicts the corresponding derivative that was attached using traditional lysine conjugation (to produce [64Cu]-lys-NOTA-Fab_{HER2}). Notably the use of DiPODS greatly increased the binding fraction. Likewise, column 3 depicts the target-binding fraction of [68Ga]-DiPODS-NOTA-Fab_{HER2}. Column 4 depicts the corresponding derivative that was attached using traditional lysine conjugation (to produce [68Ga]-lys-NOTA-Fab_{HER2}). Once again, the use of DiPODS greatly increased the binding fraction.

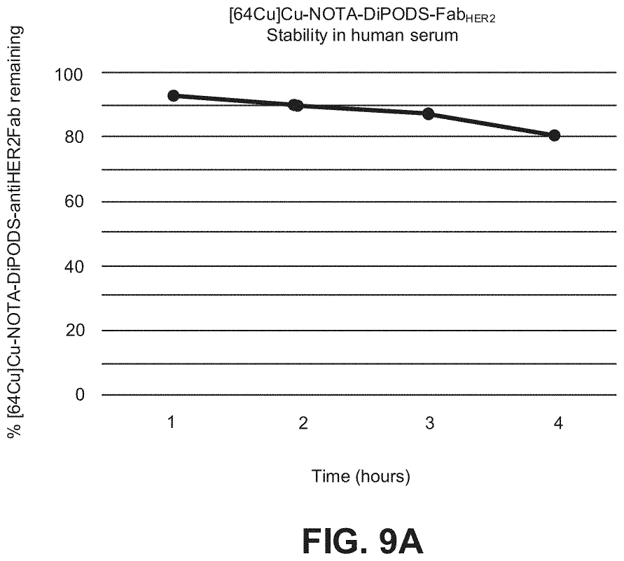

FIG. 6A shows iTLC radioanalysis of [68Ga]-DiPODS-NOTA-Fab_{HER2}. FIG. 6B shows a corresponding analysis of [64Cu]-DiPODS-NOTA-Fab_{HER2}. These results show the radioactivity is located solely within the bioconjugate and no free radionucleotide is detected.

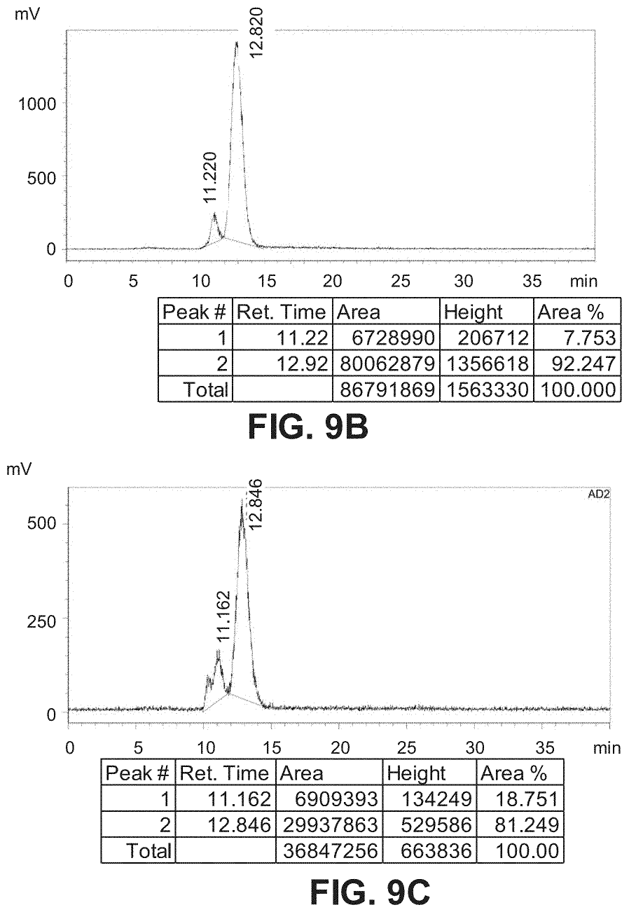

FIG. 7A shows a SEC-HPLC radioanalysis of [68Ga]-DiPODS-NOTA-Fab_{HER2} by UV-detection. FIG. 7B shows a corresponding analysis of the same compounds by radio-detection.

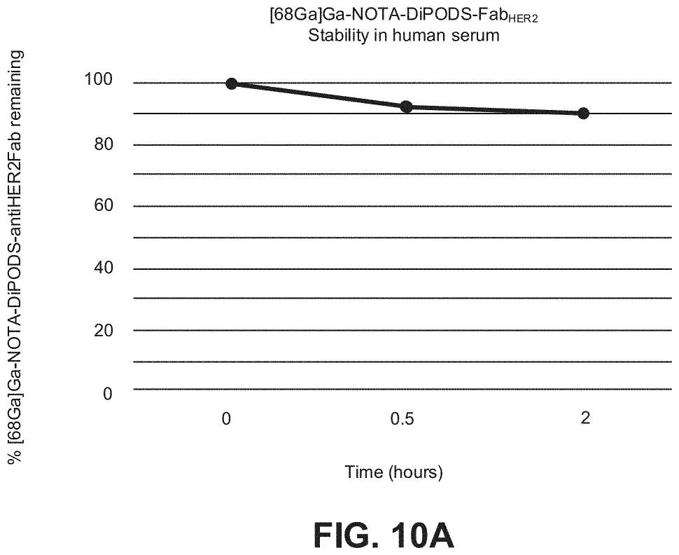

FIG. 8A shows a SEC-HPLC radioanalysis of [64Cu]-DiPODS-NOTA-Fab_{HER2} by UV-detection. FIG. 8B shows a corresponding analysis of the same compounds by radio-detection.

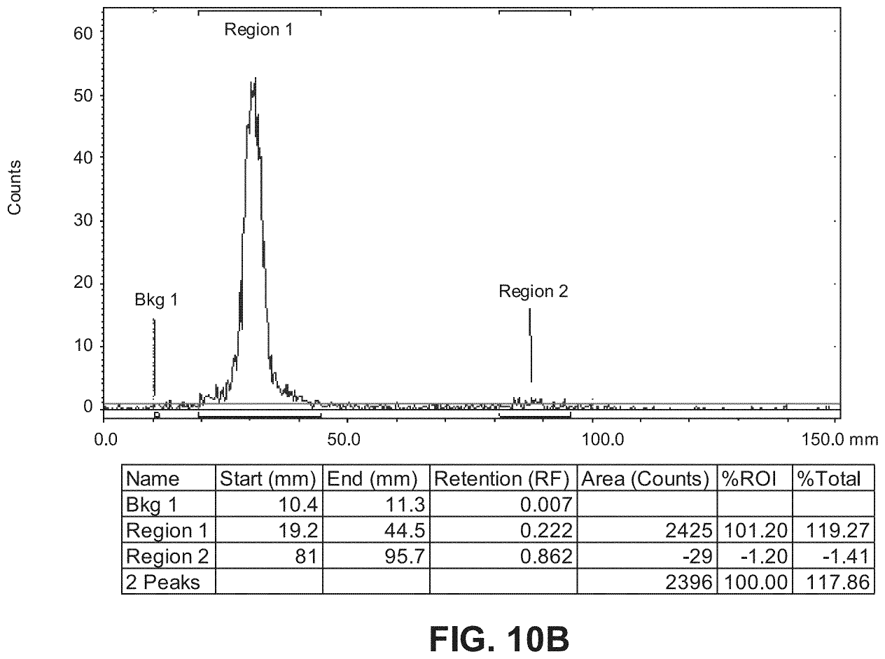

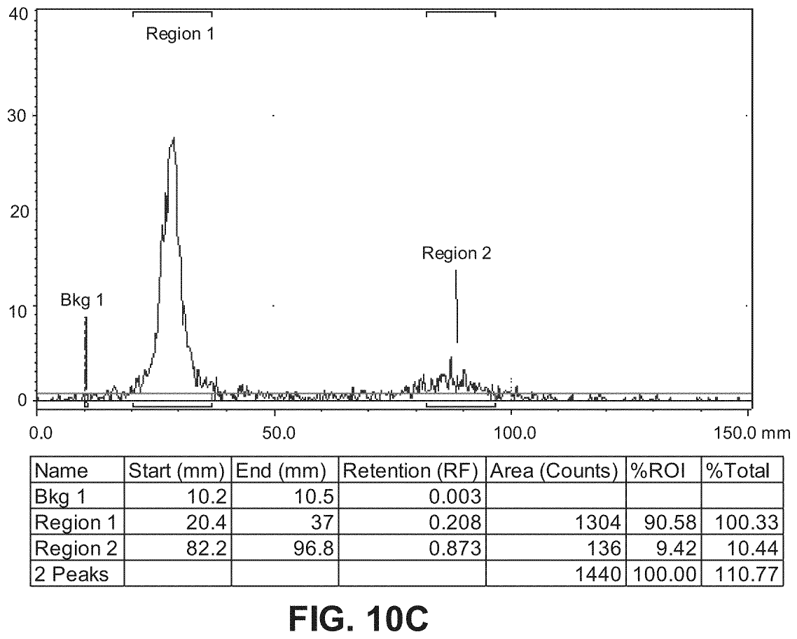

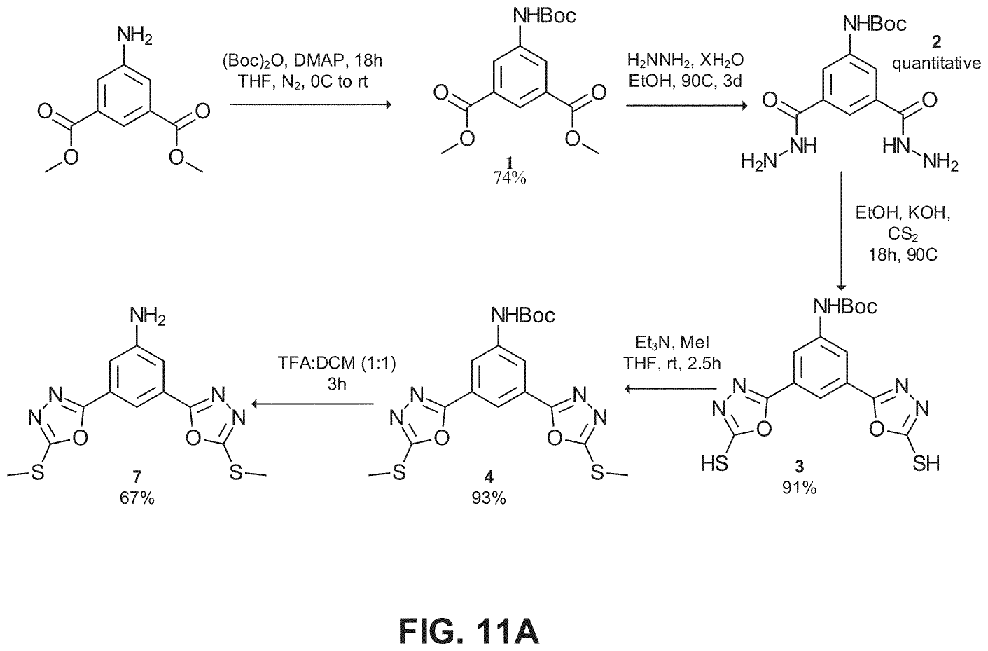

FIG. 9A is a graph depicting the stability of 64[Cu]-DiPODS-NOTA-Fab_{HER2} in human serum at 37° C. over four hours. The results show the bioconjugate is stable over the observed time period. FIG. 9B and FIG. 9C are radiochemical purity (%) assays of the same compound monitored by radio-iTLC at t=0 min (FIG. 9B) and at t=4 h (FIG. 9C) directly after incubation in human serum. The consistent results over the observed time period demonstrates the stability of the bioconjugate.

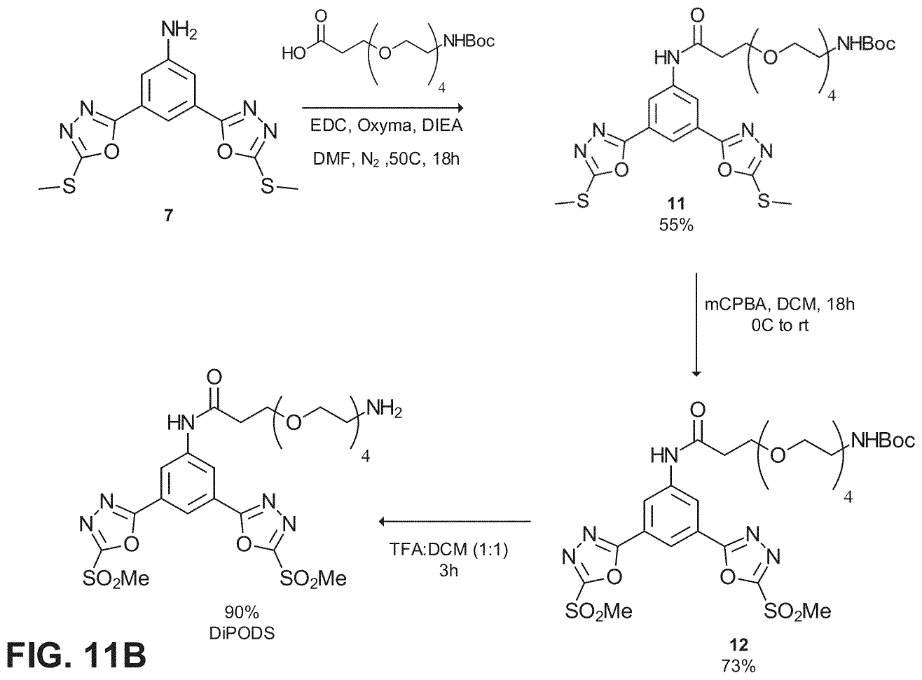

FIG. 10A is a graph depicting the stability of 68[Ga]-DiPODS-NOTA-Fab_{HER2} in human serum at 37° C. over two hours. The results show the bioconjugate is stable over the observed time period. FIG. 10B and FIG. 10C are radiochemical purity (%) assays of the same compound monitored by radio-iTLC at t=0 min (FIG. 10B) and at t=2 h (FIG. 10C) directly after incubation in human serum. The consistent results over the observed time period demonstrates the stability of the bioconjugate.

Synthesis and Characterization.

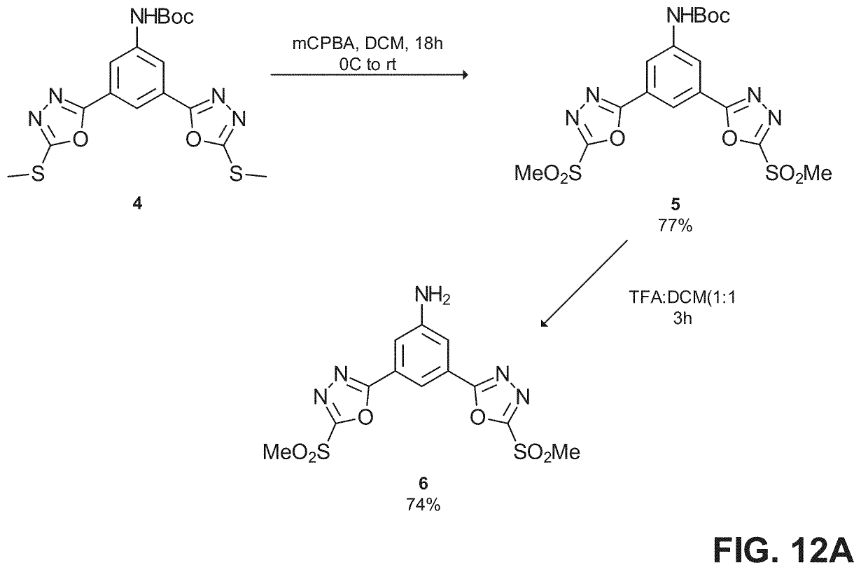

DiPODS was prepared in eight synthetic steps with good to high yield at each step (FIG. 11A and FIG. 11B). The synthesis began with the Boc-protection of aminoisophthalate, which followed a published procedure with some minor alteration. The Boc-protection was performed under nitrogen atmosphere overnight and produced compound 1 with 74% yield after purification. Surprisingly, the ¹H NMR spectrum of the crude mixture of compound 1 revealed three sets of signals for all functional groups except for the proton of the secondary amine, which was represented by a single broad peak in the ¹H NMR spectra (vide infra). While a combination of normal-phase chromatography and precipitation facilitated the partial separation of these products, all three revealed the same molecular weight by mass spectrometry, suggesting that they are conformers of 1 (for further exploration of this phenomenon, see below). The crude mixture of 1 was then treated with hydrazine hydrate and, somewhat surprisingly, produced 5-amino isophthalic dihydrazide 2 in quantitative yield. This intermediate was subsequently treated with ethanol, KOH, and carbon disulfide to create phenyl-bis(oxadiazole thiol) 3 in 91% yield. Next, the methylation of 3 using methyl iodide generated the bis(methyl thioether) 4 in near-quantitative yield.

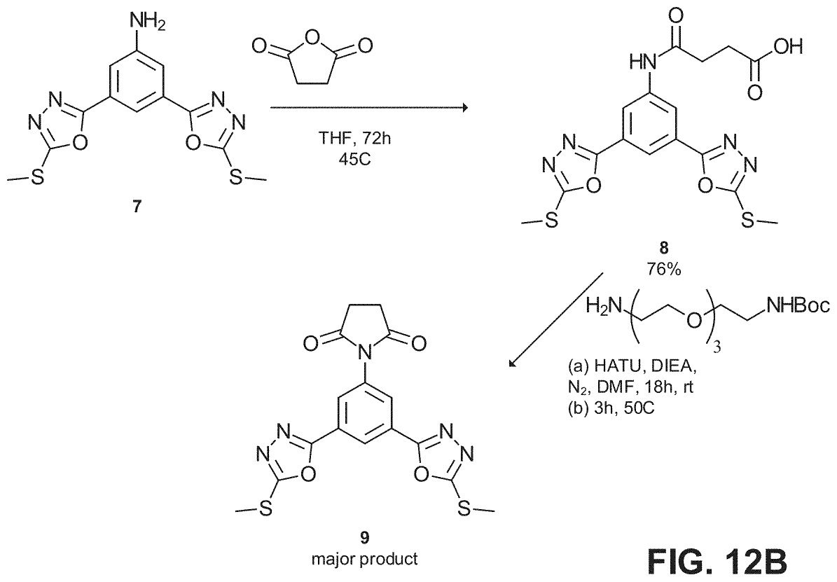

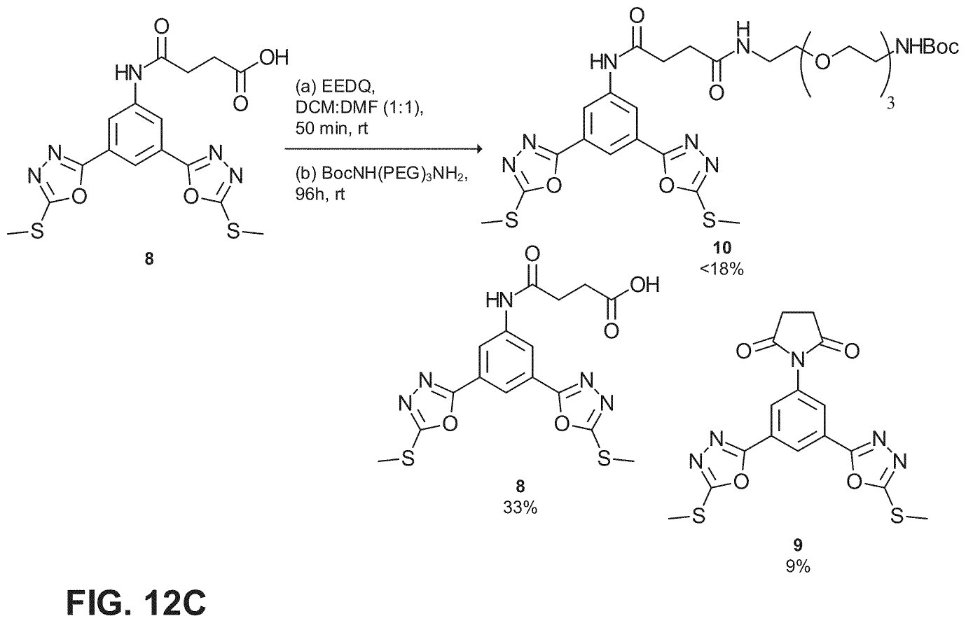

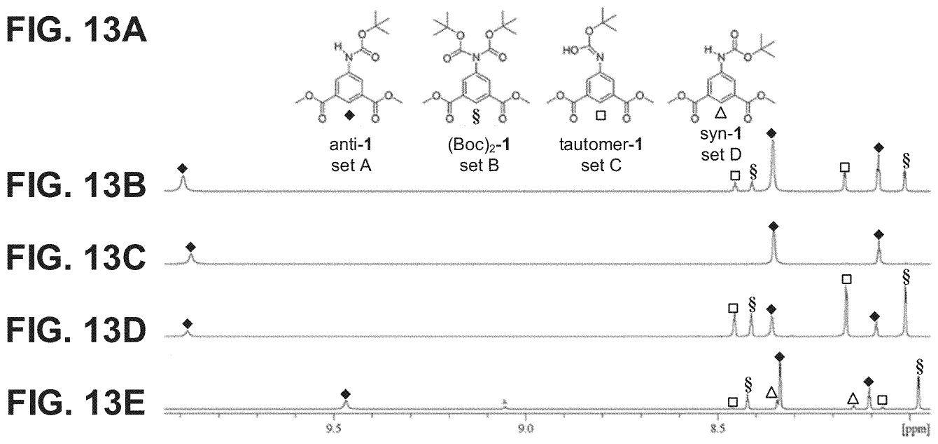

In a first attempt, bis(methyl thioether) 4 was directly oxidized via meta-chloroperoxybenzoic acid (mCPBA) to form the bis(methyl sulfonyl) 5 followed by Boc-deprotection to form compound 6 (FIG. 12A). The plan was to use compound 6 in a coupling reaction with a carboxylic acid-bearing poly(ethylene glycol) (PEG) chain. However, several attempts at this peptide coupling reaction failed or resulted in unacceptably poor yields. Aryl amine groups are notoriously poor nucleophiles, and the reactivity of the aryl-amine in compound 6 is believed to be reduced even further by the electron-withdrawing methyl sulfonyl substituents.

Methyl thioether groups are less electron-withdrawing than the methyl sulfonyl substituents. Following this logic, the coupling reaction was performed prior to the formation of the methyl sulfonyl moieties with the hope that this version of the aryl-amine had enhanced nucleophilicity. To this end, compound 4 was first deprotected in quantitative yield to produce 7. Subsequently, in the first attempt at coupling, 1-[bis(dimethylamino)methylene]-1H-1,2,3-triazolo-[4,5b]pyridinium 3-oxide (HATU) was used alongside N,N-diisopropylethylamine (DIEA). These conditions yielded <15% of the desired product, a result that mass spectrometry analysis suggested is related to the degradation of the starting materials. In response, DIEA was then swapped for a milder base-2,4,6-trimethylpyridine (TMP)—and the reaction was attempted at room temperature as well as 50° C., yet both attempts proved unsuccessful. The synthetic strategy was changing by reversing the coupling chemistry by transforming the aryl-amine into a carboxylic acid via the reaction of 7 with succinic anhydride to form 8 (FIG. 12B).

With compound 8 now containing a carboxylic acid, a peptide coupling reaction was attempted with a mono-Boc-protected bisamino-PEG chain, but the use of HATU and DIEA at both room temperature and 50° C. resulted in an unwanted cyclization and the formation of compound 9—a clear dead end—as the major product. This same transformation was then attempted using N-ethoxycarbonyl-2-ethoxy-1,2-dihydroquinoline (EEDQ) as an alternative coupling reagent (FIG. 12C).

Disappointingly, this reaction resulted in the recovery of nearly 33% starting material as well as two products: the cyclized phenyl succinimide 9 (9% yield) and the desired product 10 (<18% yield).

To continue efforts to search for a higher yielding route forward, bis(methyl thioether) 7 was used as a starting point to test a new set of peptide coupling conditions: oxyma with 1-ethyl-3-(3-(dimethylamino)propyl)carbodiimide (EDC) (FIG. 11A and FIG. 11B). At room temperature, this reaction yielded <10% of the desired product (compound 11), with mass spectrometry revealing the presence of unreacted starting material 7 as well as the O-acylisourea-activated EDC intermediate. Suspecting that the EDC intermediate was trapped in a step with an energy barrier that was impassable at room temperature, we repeated the same reaction at 50° C. This time, the PEGylated product 11 was obtained in 55% yield. To complete the sequence, 11 was then oxidized with mCPBA to create bis(methylsulfonyl) 12 in 73% yield, and—finally—compound 12 was deprotected to provide DiPODS (DiPODS-PEG4-NH₂) in ˜90% yield. This synthesis was concluded with an 8-step synthetic route to produce DiPODS with a cumulative yield of ˜15%. As synthesized, DiPODS is modular and can be coupled to any number of different bifunctional chelators, dyes, or other payloads. The primary amine of DiPODS can be reacted with a number of electrophilic bioconjugation reagents such as activated esters or phenyl-isothiocyanates.

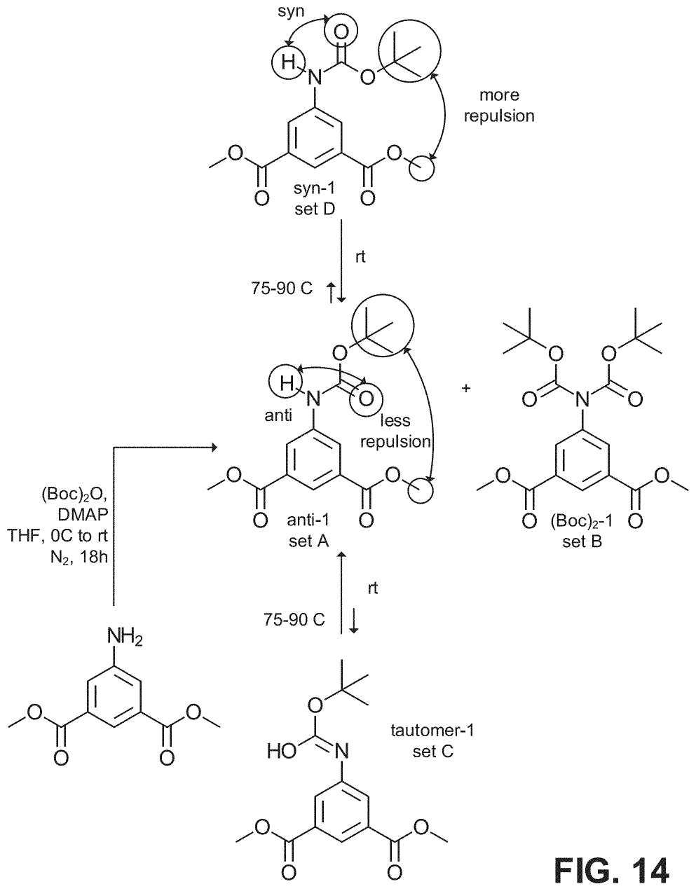

Variable Temperature NMR. The ¹H NMR of the crude mixture of 1 revealed a mixture presumed to be composed of conformers (FIG. 13A and FIG. 13B). The ¹H NMR spectrum contained three sets of signals—sets A, B, and C—for each functional group, with the exception of the Boc-protected amine, which produced a single broad peak (FIG. 13B). Curiously, the use of this mixture—without any separation—resulted in the formation of compound 2 in quantitative yield (FIG. 11A and FIG. 11B).

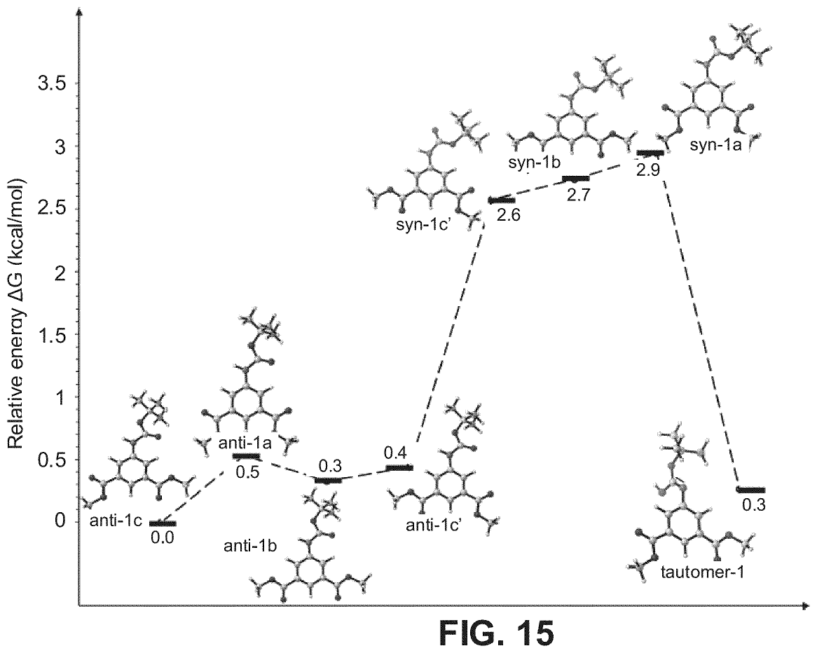

In an attempt to separate and identify the components of the crude product mixture, it was dissolved in warm DCM and stored at −20° C. overnight. The first attempt at precipitation produced a shiny white precipitate that was separated from the mother liquor via filtration and dried under high vacuum. The ¹H NMR of this white precipitate displayed only one set of signals—set A—for all the functional groups, including the proton of the secondary amine (FIG. 13C). The isolation of pure set A, and the following investigation of the remaining mother liquor mixture by VT-NMR strongly suggests that compound 1—like many other carbamate-bearing molecules—exists as syn- and anti-rotamers. The anti-rotamers of compound 1 are more energetically favored due to less steric hindrance between the tert-butyl group and the ester group (FIG. 13A and FIG. 14; for a more detailed discussion, see the section entitled Computational Studies). Therefore, the signals of set A were assigned to a mixture of the anti-rotamer of compound 1. To be more specific, while the anti-rotamer configuration of the Boc group remains constant, the two methyl ester groups can rotate freely, creating a subset of conformers for each of the syn- and anti-rotamers (i.e., subconformers).

After isolating the precipitate from the crude product mixture, the solvent was removed from the mother liquor, and the solid residue was subjected to several more rounds of precipitation. After each round, the precipitate was isolated, and each time it was found via ¹H NMR to be predominantly composed of the anti-rotamer (set A). Following several rounds of precipitation, the aggregate mother liquor was concentrated under vacuum and found via ¹H NMR to contain both sets B and C as well as a small amount of set A (FIG. 13D). In order to better understand the NMR spectrum of the product mixture of compound 1, a series of NMR spectra were collected at different temperatures. Two NMR samples were prepared from the components of crude compound 1. The first contained only the precipitate, i.e., the anti-rotamers (FIG. 13C, set A). The second contained the mixture isolated from the mother liquor following precipitation (13D). The latter is composed mostly of the compounds responsible for sets B and C but also some of the anti-rotamer (set A). A more detailed explanation of the VT-NMR experiments and assignments can be found in the section entitled Materials and Methods of U.S. provisional patent application No. 63/216,672.

Ultimately, set B was attributed to a doubly Boc-protected version of compound 1 based on the integration ratio between the methyl ester (6) and tert-butyl (18) protons, as well as the presence of a tertiary amine group with no proton signal. High resolution mass spectrometry subsequently confirmed this assignment. As removing the first of two Boc protecting groups is easier than the second, the doubly protected compound (set appears to be converted to compound 1 at elevated temperatures (VT NMR, FIG. 13E). Set C, in contrast, has an integration ratio of 6:9 between the methyl ester (6) and tert-butyl (9) protons, confirming that the compound responsible for these peaks has a single Boc group. However, no proton associated with the amine was observed. Furthermore, upon heating to 90° C. set C disappeared almost entirely. This phenomenon can be explained by a tautomerization reaction involving the transfer of a proton from the amine to the neighboring oxygen (FIG. 14). The assignment of set C as a tautomeric form of set A would explain why the integration of the former matches that of the latter except for the absence of the proton from the amine group. In the end, these NMR data permit us to deconvolute the constituents of the original compound 1 product mixture: an anti-rotamer of compound 1 (anti-1, set A), a doubly Boc-protected variant of compound 1 [(Boc)2-1, set B], and an imidic acid tautomer of compound 1 (tatomer-1, set C) (FIG. 14). These findings also explain how a crude mixture of 1 containing all of these components was reacted with hydrazine hydrate and produced 5-amino isophthalic dihydrazide 2 in quantitative yield.

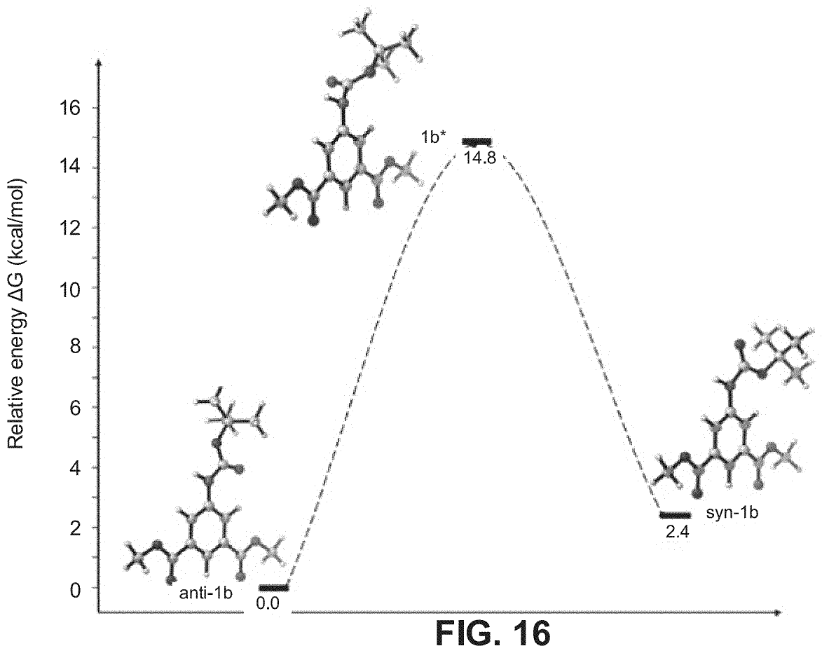

Computational Studies. Computational investigation of the isomers of compound 1 supports the assignments made based on the VT-NMR data. The calculated Gibbs free energies of the rotamers of compound 1 revealed that the anti-rotamers are favored by ˜2.0 kcal/mol (FIG. 15). This figure shows the calculated structures of two groups of rotamers and a tautomer of compound 1. The first group of rotamers includes four configurations of anti-rotamers (anti-1a, anti-1b, anti-1c, and anti-1c′) with energies similar to each other and to tautomer-1. The second group—which consists of three configurations of syn-rotamers (syn-1a, syn-1b, and syn-1c′) with similar energies—lies ˜2.0 kcal/mol higher than the anti-rotamers and the tautomer. The energy difference between the four anti-rotamers is very small (˜0.5 kcal/mol), suggesting they can interconvert at room temperature. This explains why they all manifest as a single set of signals (set A) in the ¹H NMR spectrum of compound 1 despite having different point group symmetries. Despite the calculated similarity in energy between the set A anti-rotamers and the imidic acid tautomer-1 (Set C), they do not appear to interconvert at ambient temperature (13B). This suggests that a higher energy transition state must be passed for conversion, which is supported by the disappearance of the tautomer (set C) at elevated temperatures.

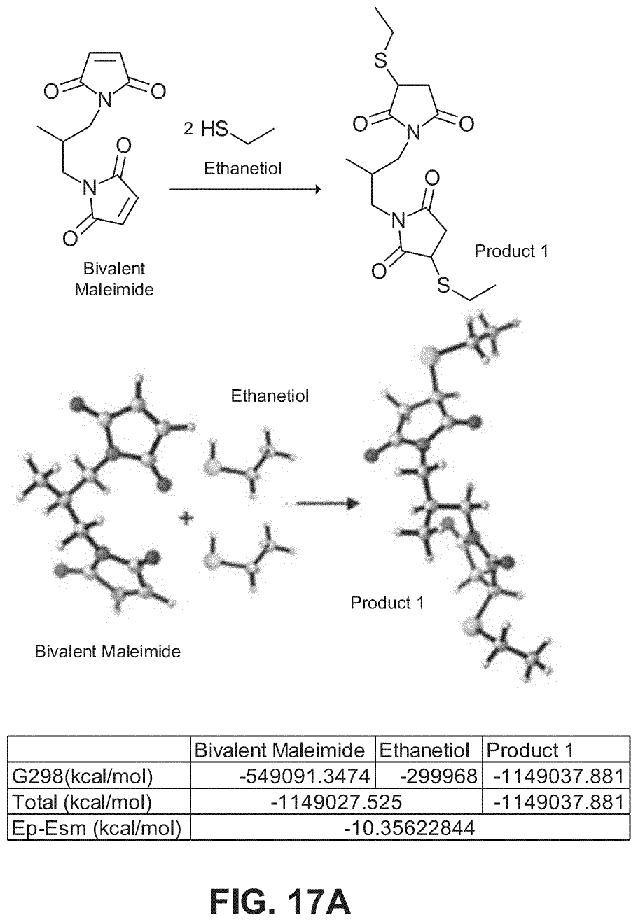

The interconversion between the anti-(set A) and syn-(set D) rotamers occurs via the rotation of the Boc group attached to the amine. In order to further understand this process, the transition state was calculated for one such rotation between anti-1b and syn-1b (FIG. 16). To identify the transition state (1b*), the anti-1b rotamer, and the dihedral angle of interest was varied in a stepwise fashion toward that of the syn-1b rotamer using Spartan 14 software. The structure with the highest energy was carried forward for optimization as the transition state in Gaussian 16. The harmonic vibrational frequencies showed only one imaginary frequency, corresponding to the desired transition. The energy difference between the transition state and the anti-1b rotamer is substantial (˜15 kcal/mol) and thus might not be overcome at room temperature, depending on other conditions such as solvent (FIG. 16). One way to overcome this large energy barrier, however, is via heating, which could explain the formation of a separate set of ¹H NMR signals (set D) at elevated temperatures. It is important to note that the energy difference between the syn-rotamers is also small (˜0.3 kcal/mol), suggesting that they can interconvert easily at elevated temperatures and thus explaining their appearance as a single set of peaks in the ¹H NMR spectra.

Taken together, the aforementioned NMR and computational studies helped deconvolute the mixture of components formed when synthesizing compound 1. Furthermore, these data help explain how this mixture of anti-rotamers, tautomer-1, and a doubly Boc-protected variant of compound 1 can react together to form compound 2 in near-quantitative yield: the elevated temperature of the reaction—90° C. for 3 days—would overcome any rotational energy barriers and allow for the production of compound 2 in quantitative yield.

The desired applications that use DiPODS require it to react in a predictable and reproducible manner with two thiols. For example, if the reactivity were to be different for two rotamers/isomers of DiPODS, this could become an important physical property to understand. From these investigations, the rotamer behavior appears to be largely the result of the Boc-protected amine and therefore not likely to be an issue for the final DiPODS compounds. Further, the imidic acid tautomer (set C) is not likely to form in DiPODS itself or its derivatives, as the pKₐ of the amide in these final conjugates is higher than that of the Boc-protected carbamate (which forms set C).

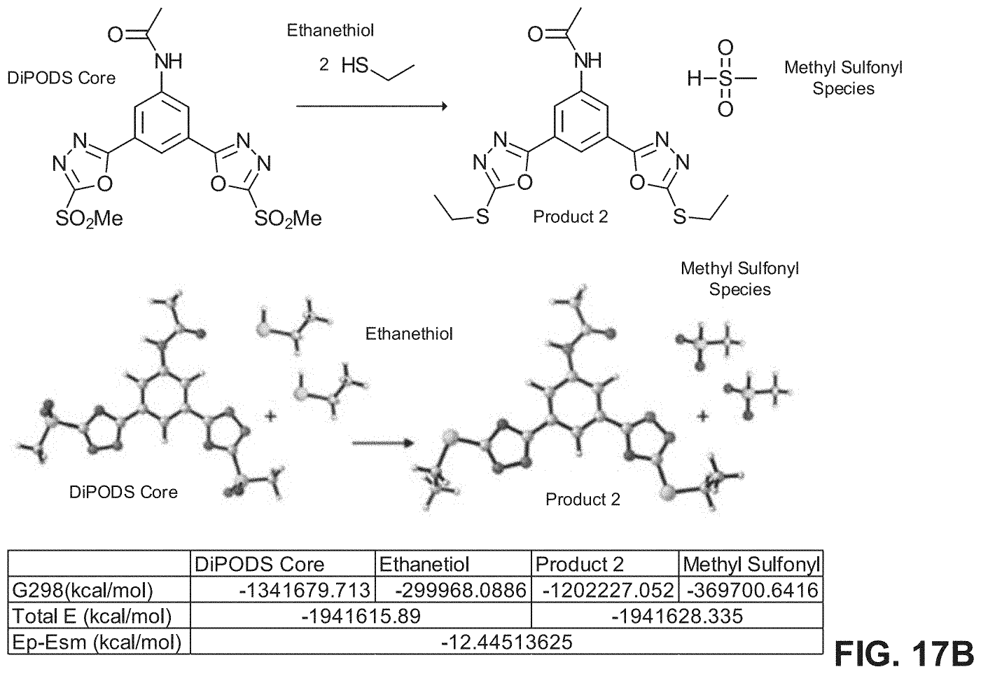

Computational methods were also used to compare the thermodynamic stability of the conjugation product formed by DiPODS to those formed by a bivalent maleimide, a monovalent maleimide, and a monovalent PODS. To this end, ethanethiol was employed as a simple surrogate substrate, and the total energy of the final product(s) was compared to the total energy of the starting materials using the UAHF model for improved solvent modeling (FIG. 17A and FIG. 17B). Since all of the reactions were modeled as isodesmic, the difference in total energy—i.e., Gibbs free energy—between the reactants and products in each case, could be calculated, thereby enabling a comparison between the net change in thermodynamic stability of each transformation. The ligation between ethanethiol and the monovalent maleimide resulted in a net Gibbs free energy change of −5.3 kcal/mol, while that between the same substrate and the monovalent PODS is slightly more stabilizing, with a net change of −5.6 kcal/mol. Not surprisingly, the divalent reagents created larger changes in free energy. More specifically, the reaction of the bivalent maleimide resulted in a change in Gibbs free energy of −10.3 kcal/mol, while the ligation between DiPODS and a pair of ethanethiol substrates provided an even greater gain in stability: −12.4 kcal/mol. While an extra ˜2.1 kcal/mol of stabilization does not represent a dramatic improvement, it—combined with the irreversibility of the DiPODS-based conjugation—certainly suggests that DiPODS-based conjugates will be more stable than their bismaleimide-based analogues both in vitro and in vivo.

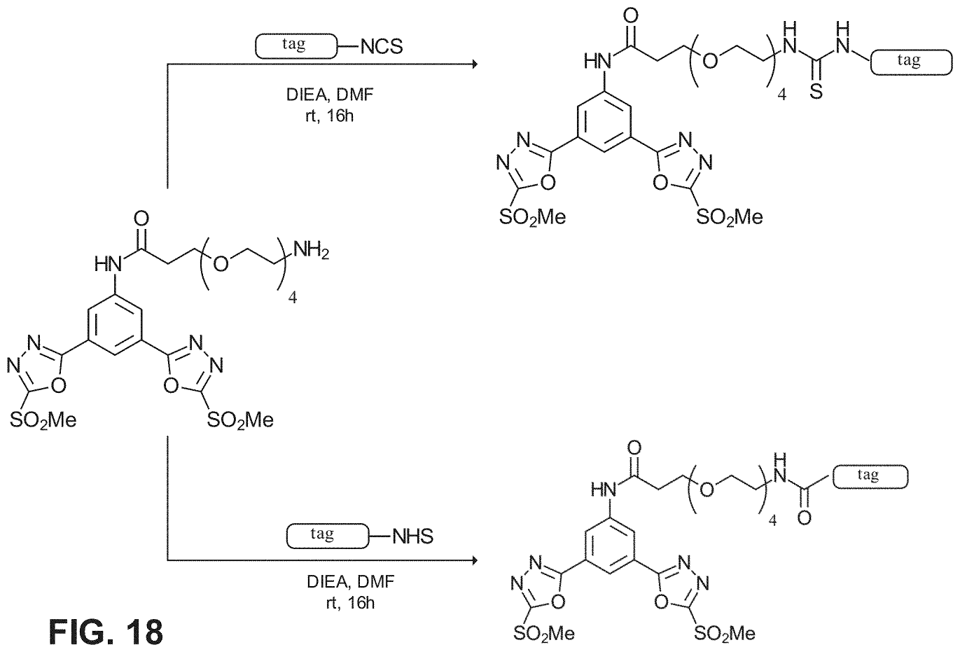

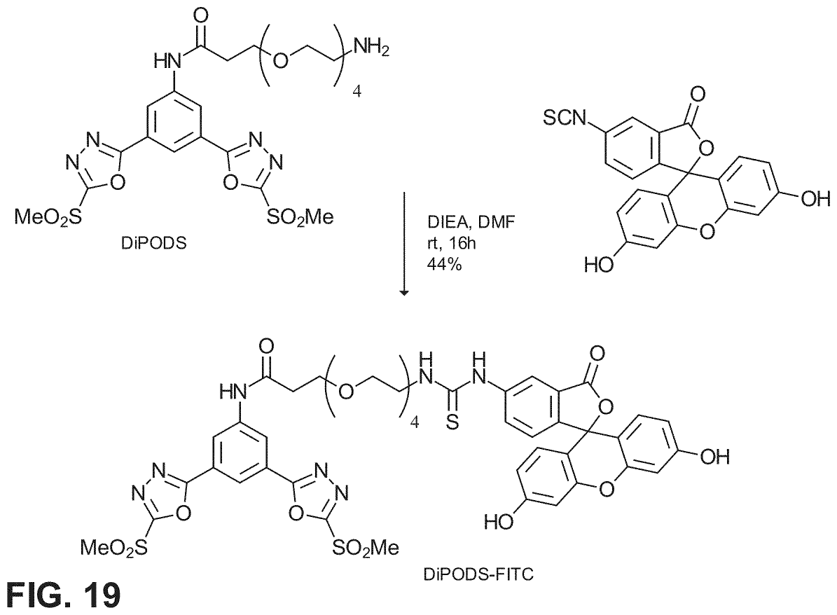

Synthesis of a Fluorophore-Bearing Variant. DiPODS was designed to be modular, as its reactive primary amine facilitates the coupling of cargoes such as chelators, dyes, and toxins. FIG. 18 provides a general synthetic scheme for attaching various tags. In order to facilitate proof-of-concept reactivity and bioconjugation experiments, a fluorescein-bearing variant of DiPODS-DiPODS-FITC—was prepared via the reaction of DiPODS-PEG4-NH₂ with fluorescein isothiocyanate in the presence of DIEA (FIG. 19)

Reactivity with a Model Thiol. N-Acetyl-L-cysteine methyl ester was used as a model thiol to evaluate the reactivity of DiPODS-FITC. To this end, DIPODS-FITC was incubated at room temperature with 10 equiv of N-acetyl-L-cysteine methyl ester and 5 equiv of a mild reducing agent, tris(2-carboxyethyl)-phosphine (TCEP). The progress of the reaction was interrogated via LC-MS 5 min after mixing, and quantitative conversion to DiPODS-FITC-Cys2 was observed.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter consisting of: 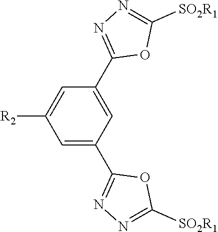
wherein R₁ is methyl, ethyl or propyl and R₂ is a fluorescent label, a metal chelator or a click-chemistry synthon.

2. The composition of matter as recited in claim 1, wherein R₂ comprises 1,4,7-triazacyclononane-N,N′,N″-triacetic acid.

3. The composition of matter as recited in claim 1, wherein R₂ is a metal chelator, the composition further comprising a chelated metal ion.

4. The composition of matter as recited in claim 1, wherein R₂ comprises 1,4,7-triazacyclononane-N,N′,N″-triacetic acid (NOTA).

5. The composition of matter as recited in claim 1, wherein R₂ comprises NHS-Fluorescein.

6. The composition of matter as recited in claim 1, wherein R₂ is an antibody-based fluorescent label.

7. The composition of matter as recited in claim 1, wherein R₂ comprises a click-chemistry synthon selected from a group consisting of trans-cycloctyl (TCO) derivatives and N3.

8. The composition of matter as recited in claim 1, wherein R₂ comprises 1,4,7,10-Tetraazacyclododecane-1,4,7,10-tetraacetic acid (DOTA).

9. The composition of matter as recited in claim 1, wherein R₂ is a green fluorescent dye.

10. A method for labeling a substrate, the method comprises steps of:
exposing a label to a substrate that comprises two cysteine residues, wherein the label comprises: 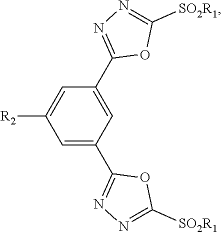
wherein R₁ is methyl, ethyl or propyl and R₂ is a fluorescent label, a metal chelator or a click-chemistry synthon;
permitting the label to covalently bind to the two cysteine residues of the substrate, thereby labeling the substrate.

11. The method as recited in claim 10, wherein R₂ comprises 1,4,7-triazacyclononane-N,N′,N″-triacetic acid.

12. The method as recited in claim 10, wherein R₂ is a metal chelator, the composition further comprising a chelated metal ion.

13. The method as recited in claim 10, wherein R₂ comprises 1,4,7-triazacyclononane-N,N′,N″-triacetic acid (NOTA).

14. The method as recited in claim 10, wherein R₂ comprises NHS-Fluorescein.

15. The method as recited in claim 10, wherein R₂ is an antibody-based fluorescent label.

16. The method as recited in claim 10, wherein R₂ is a click-chemistry synthon selected from a group consisting of trans-cycloctyl (TCO) derivatives and N3.

17. The method as recited in claim 10, wherein R₂ comprises 1,4,7,10-Tetraazacyclododecane-1,4,7,10-tetraacetic acid (DOTA).

18. The method as recited in claim 10, wherein R₂ is a green fluorescent dye.

19. A composition of ma ter consisting of: 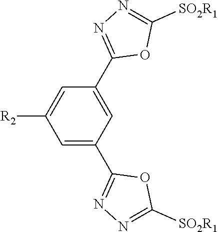
wherein R₁ is methyl, ethyl or propyl and R₂ is

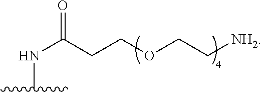

20. The composition of matter as recited in claim 19, wherein R₁ is methyl.
